(19) United States
(12) Patent Application Publication
(10) Pub. No.: US 2024/0398088 A1
(43) Pub. Date: Dec. 5, 2024

(54) HAIRCARE APPLIANCE

(71) Applicant: Dyson Technology Limited, Malmesbury, Wiltshire (GB)

(72) Inventors: Nathan Haruna Waters IMHASLY, Bath (GB); Thomas Alexander CARLYLE, Chippenham (GB); Maurizio BALLARATI, Swindon (GB); Henry Oliver Tate MILLS, Bristol (GB); Bartosz SLAK, Chippenham (GB); Robert Mark Brett COULTON, Bristol (GB)

(21) Appl. No.: 18/718,468

(22) PCT Filed: Dec. 13, 2022
(86) PCT No.: PCT/GB2022/053199

§ 371 Date: Jun. 11, 2024

(30) Foreign Application Priority Data
Dec. 23, 2021 (GB) 2118886.7

(51) Int. Cl. A45D 20/12; A45D 1/04; A45D 1/28; G01N 21/3554

(52) U.S. Cl. CPC A45D 20/12; A45D 1/04; A45D 1/28; G01N 21/3554

(57) ABSTRACT

A haircare appliance is provided for detecting a hair moisture content. The haircare appliance includes a processing element, at least one light emitting element emitting at least two wavelengths, and a sensor element, wherein the light emitting element is adapted to irradiate a portion of hair to be treated. The sensor element is adapted to receive a sensor reading from an acquisition region of the irradiated hair and generate a sensor signal dependent on the sensor reading and includes a spectral sensor element. The processing element is adapted to determine a moisture content of at least the irradiated portion of the hair from the sensor signal.

### TECHNICAL FIELD

The present disclosure relates to haircare appliances. In particular, the present disclosure relates to a haircare appliance arranged for detecting a hair moisture content and to adapting the mode of operation of a haircare appliance to a detected hair moisture content. Further in particular, the present disclosure relates to a haircare appliance that provides a flexible internal placement of a radiation source or a measurement sensor and connecting the same to an acquisition region for acquisition of a sensor measurement or to a treatment region to which radiation is provided.

### BACKGROUND

The drying of hair has always been a significant part of one's daily or weekly routine. In order to protect hair to be dried and to provide an exceptional drying experience, it may be beneficial to determine the moisture content of hair to be dried. In particular, it may be especially beneficial to obtain information on current moisture content of hair in the course of drying the hair. In order to obtain a current moisture content, a substantially continuous, real-time determination of a moisture content is particularly preferable. In other words, it is particularly beneficial to obtain a current moisture content while in the process of drying hair.

Haircare appliances may be required to emit and/or receive electromagnetic radiation, e.g., light, to enable various light-based product features like sensing systems using techniques such as spectroscopy, imaging, reflectometry, measuring hair moisture levels, hair temperature, or distance measurements. Further, hair, scalp, or skin treatments may be implemented that irradiate part of the body of a user with specific wavelengths of light, e.g., red-light therapy (RLT) or low-level laser therapy (LLLT) for hair loss. Still further, sterilisation functionality may be provided by using electromagnetic radiation having a specific wavelength, e.g., UVC with 100-280 nm to kill microorganisms. Finally, a haircare appliance may provide visual feedback or implement user interface indicators, e.g., LED colour variation, to represent different machine states.

One particular implementation of such a sensing system may be in the context of hair moisture detection solutions, which may be inaccurate and may not necessarily provide the information necessary that translates into an exceptional styling experience for a user. Many current moisture sensing solutions do not provide a moisture content value that is sufficiently reliable or accurate in order to base a styling decision thereon. In particular, current moisture sensing technology may be impacted by a certain colour of the hair to be dried, a distance between a moisture sensor and the target hair, ambient temperature or humidity, or simply by the inability to detect hair presence, thereby resulting in an inaccurate moisture determination and thus a diminished styling experience.

Not knowing a current moisture content may lead to an over drying of hair, a prolonged drying experience or, even worse, in damage to the hair. Likewise, not knowing a current moisture content may lead to an under drying of hair, which could lead to poor styling experience.

Thus, there may be a need for an accurate current moisture content determination in order to provide an exceptional styling experience to a user.

Further, there may be a need to arrange a sensor element for accurate moisture content determination so as to facilitate a reliable and, in particular, repeatable determination procedure.

Still further, there may be a need to influence a mode of operation of a haircare appliance dependent on the determined moisture content of hair to be dried and/or guide a user of the haircare appliance to heighten a styling experience.

### SUMMARY

At least one such need may be met by the subject-matter of the independent claims. Preferred embodiments are provided in the dependent claims and are explained in detail in the following description.

The present invention relates to detecting the moisture content of a hair in a reliable manner to provide an exceptional styling experience to a user of a haircare appliance.

According to a first aspect of the present disclosure, there is provided a haircare appliance, comprising a main body comprising a blower for generating an airflow, wherein the main body comprises an attachment region adapted for connecting an accessory, and an accessory, comprising an element for obtaining at least one sensor reading at an acquisition region at or around the accessory, wherein information related to the sensor reading is obtained at the acquisition region, wherein the accessory is attachable to and/or detachable from the main body at the attachment region, wherein the accessory, when attached to the main body, is adapted to receive the airflow from the blower, and wherein the accessory is adapted to discharge the received airflow towards the hair of a user, wherein the information related to the sensor reading is transmitted from the acquisition region to a sensor element, and wherein the information is transmitted wirelessly.

According to a second aspect of the present disclosure, there is provided a haircare appliance, comprising a processing element, a light emitting element, and a sensor element, wherein the light emitting element is adapted to irradiate hair to be treated, wherein the sensor element is adapted to receive a sensor reading from an acquisition region of the irradiated hair and generate a sensor signal dependent on the sensor reading, wherein the processing element is adapted to determine a moisture content of at least a portion of the irradiated hair from the sensor signal, wherein the determination of the moisture content is further dependent on at least one parameter out of the group consisting of a temperature of the hair, a distance between the haircare appliance and the hair, a hair presence, an environmental condition, an ambient temperature, an ambient air pressure, an ambient light level, an ambient humidity, a hair colour, a hair melanin content, and a user input.

According to a third aspect of the present disclosure, there is provided a haircare appliance, comprising a processing element, at least one light emitting element emitting at least two wavelengths, and a sensor element, wherein the light emitting element is adapted to irradiate hair to be treated, wherein the sensor element is adapted to receive a sensor reading from an acquisition region of the irradiated hair and generate a sensor signal dependent on the sensor reading, wherein the sensor element is a spectral sensor element, and wherein the processing element is adapted to determine a moisture content of at least a portion of the irradiated hair from the sensor signal.

According to a fourth aspect of the present disclosure, there is provided a haircare appliance comprising a processing element, a light emitting element, a sensor element, and a main body comprising a blower for generating an airflow and the sensor element, wherein the main body comprises an attachment region adapted for connecting an accessory, wherein the light emitting element is adapted to irradiate hair to be treated, wherein the sensor element is adapted to receive a sensor reading from an acquisition region of the irradiated hair and generate a sensor signal dependent on the sensor reading, wherein the processing element is adapted to determine a moisture content of at least a portion of the irradiated hair from the sensor signal, wherein the determination of the moisture content is further dependent on at least one parameter out of the group consisting of a temperature of the hair, a distance between the haircare appliance and the hair, a hair presence, an environmental condition, an ambient temperature, an ambient air pressure, an ambient light level, a relative humidity, a hair colour, a hair melanin content, humidity and a user input, further comprising an accessory wherein the accessory is attachable to and/or detachable from the main body at the attachment region, wherein the accessory, when attached to the main body, is adapted to receive the airflow from the blower, wherein the accessory is adapted to discharge the received airflow towards the hair of a user, and wherein the operation of the haircare appliance is adaptable dependent on the sensor reading and/or the determined moisture content, further in particular dependent on the type of accessory attached to the main body.

According to a fifth aspect of the present disclosure, there is provided a haircare appliance comprising a processing element, a light emitting element, a sensor element, and a main body comprising a blower for generating an airflow, wherein the light emitting element is adapted to irradiate hair to be treated, wherein the sensor element is adapted to receive a sensor reading from an acquisition region of the irradiated hair and generate a sensor signal dependent on the sensor reading, wherein the processing element is adapted to determine a moisture content of the irradiated hair from the sensor signal, wherein the determination of the moisture content is further dependent on at least one parameter out of the group consisting of a temperature of the hair, a distance between the haircare appliance and the hair, a hair presence, an environmental condition, an ambient temperature, an ambient air pressure, an ambient light level, a relative humidity, a hair colour, a hair melanin content, humidity and a user input, further comprising an accessory integrally connected with the main body, wherein the accessory is adapted to receive the airflow from the blower, wherein the accessory is adapted to discharge the received airflow towards the hair of a user, and wherein the operation of the haircare appliance is adaptable dependent on the sensor reading and/or the determined moisture content.

The haircare appliance may comprise one or more optical components for the purpose of collecting, transporting/guiding, and distributing light between an object (e.g., the hair) and the main body of a haircare appliance (e.g., a hair dryer) via an accessory, connectable to an attachment portion (e.g., a diffuser or a concentrator nozzle). These optical components may include lenses, mirrors, hollow prismatic pipes, light rods, mirrored light pipes, or fibre optics, and may transport the light by multiple specular reflections or total internal reflection. Such an element may equally be referred to in the further context of this disclosure as an element for obtaining a sensor reading or a light transport system. Such an element for obtaining a sensor reading may be understood as assisting in obtaining the sensor reading, but not necessarily generating the sensor reading itself. Likewise, the element may or may not be a sensor element. Transmitting light may in particular be understood as transporting electromagnetic energy.

The element may direct or propagate electromagnetic radiation from a radiation source/a sensor element towards the object and/or may direct or propagate electromagnetic radiation from the object to the sensor element. Preferably, the object for obtaining a sensor reading may be manufactured from a material suitably transmissive to the wavelength of light being either emitted towards the hair, reflected from the hair, or emitted from the hair due to thermal electromagnetic radiation or fluorescence. The light transport system may be spatially aligned on one side with a component on the main body of the haircare appliance for emitting and/or sensing light, e.g., a sensor element or radiation source, and the opposite side directed towards an acquisition region/irradiation region being targeted by the haircare appliance.

Using interchangeable accessories for propagating/directing the electromagnetic radiation may reduce the overall cost on each attachment. Accessories including a light transport system may likely be simpler, cheaper, and more reliable than adding a sensor element to each accessory, which may further require dedicated power and data connections. The light transport system design may be varied and/or adapted for each accessory type to provide optimal sensing performance for the associated use case. For example, the point of light emission/measurement, trajectory or focal length may be matched to the particular treatment area or acquisition region that requires illumination or the offset distance expected (e.g., rough dry=−50 mm offset, diffuser =−10 mm offset, curling barrel=0 mm offset etc.). An accessory using such a light transport system may circumvent any line-of-sight obstructions within an accessory, as it may propagate light around bent corners or tight spaces, whilst also providing efficient transmission of the photonic energy between accessory sides. Thermally sensitive components may be positioned away from the heated airflow, but their point of emission or sensing area may remain within a treatment or acquisition region, thereby ensuring that the sensed/illuminated region is the same as that being heated and dried by the airflow of the device. When the present disclosure refers to “light”, this is to be understood in the broadest sense, and should encompass electromagnetic radiation as UV radiation, IR radiation or visible light. Hair may also be understood as a fur of an animal, hair extensions or artificial hair.

The haircare appliance according to the present disclosure may likewise be one where the main body and the accessory form an integral connection, so that the main body and the accessory may not be separable. Essentially, this may correspond to a haircare appliance that has a single dedicated functionality, e.g., a hair blower, a hair curler or a hair straightener. The accessory may not be embodied prominently. E.g., in case of a hair blower, a nozzle for directing and forming an airflow may be an accessory in the sense and meaning of the present disclosure. It may in particular not be necessary, but conceivable, that an accessory is in itself an active element of the haircare appliance. The sensor element may be arranged within the interior of the haircare appliance and arranged so to receive a sensor reading or measurement signal from an acquisition region. Likewise, the sensor element itself may be arranged within the acquisition region, thereby directly receiving the sensor reading or measurement signal from the acquisition region. It is conceivable that a haircare appliance with an integrally formed accessory still comprises an object for obtaining a sensor reading, connecting a sensor element with an acquisition region as per the present disclosure.

Current hair moisture detection solutions are inaccurate and may not be able to provide information that translates into an exceptional styling experience to the user. Regularly, moisture sensing technology challenges are related to accuracy and specificity of moisture information readout that may be subject to hair colour, distance to target (sensor vs hair location/position and distance), ambient temperature change, or simply the ability to detect if a hair tress is present in the region of interest.

The present disclosure provides enhanced and accurate moisture detection by utilising further parameters when determining a moisture content of a hair. In particular, the present disclosure is employing a sensor element like a spectral sensor that operates based on measuring light absorbed by a target in near infrared region of electromagnetic spectrum. The sensor element may consist of infrared LEDs, photodiodes and embedded optical filters. In other words, a signal is sent from the sensor towards an acquisition region, where the signal interacts with the hair to be measured, which signal in turn is propagated back to the sensor element for acquisition of the sensor signal. The received sensor signal in turn is analysed and a moisture content of the hair in the acquisition region is calculated. The sensor signal may be a differential signal and may be dependent on the field of view, distance to hair, hair colour and surrounding temperature.

The moisture detection of the present disclosure utilizes light of at least two different wavelengths or different wavelength bands. The different wavelengths interact in an acquisition region with an object which comprises a target property or target chemical, and at least a part of the light that has interacted with the target property or target chemical is subsequently received by a sensor element.

The sensor element is arranged to detect light of a different wavelength or wavelength band that has interacted with the target property or target chemical. A ratio is calculated between a parameter indicative of the intensity of light detected in the first wavelength or wavelength band and a parameter indicative of the intensity of light detected in the second wavelength or wavelength band, and the presence and/or amount of the target property or target chemical may be determined from the calculated ratio. Such a target property may be, for example, a hair colour and a target chemical may be, for example, melanin.

E.g., the first wavelength or wavelength band may be in the range of about 1450 nm and the second wavelength or wavelength band may be in the range of about 1920 to 1960 nm. A ratio of two measurements, each relating to one of the two wavelengths or wavelength bands may be calculated. For each of the two wavelengths or wavelength bands, water may absorb the radiation at differing amounts. Ratioing the two measurements allows to determine the water content, and thus the moisture content of the hair currently measured.

The moisture content may be determined using the Beer Lambert Law based on the derivation of ratios. According to the Beer Lambert Law (EQ1), the absorbance of a sample may be defined as the logarithm of the ratio of incident to transmitted radiant power through a sample. Alternatively, for samples which scatter light, absorbance may be defined as the negative logarithm of one minus absorptance, as measured on a uniform sample.

$$A = ε c l \qquad EQ1$$

with

A: absorbance

ε: molar absorption coefficient

c: molar concentration

I: optical path length

EQ2 and EQ3 define the light intensity after reflection of incident EM radiation, i.e., light of a defined wavelength or wavelength band by the target object, e.g., hair. Filt1 and filt 2 refer to the respective wavelength or wavelength band. E.g., filt1 may be EM radiation in the range of 1450 nm while filt2 may be in the range of 1920 nm to 1950 nm.

$${I}_{filt 1} = {I}_{0} 1 {0}^{- {μ}_{filt 1} c l} \qquad EQ2$$

$${I}_{filt 2} = {I}_{0} 1 {0}^{- {μ}_{flit 2} c l} \qquad EQ3$$

with

μ_{filt1}: water attenuation coefficient for a given filter

μ_{filt2}: water attenuation coefficient for a given filter, differing from μ_{filt1}

C: water concentration

I: optical path length

I₀: a broader band light source comprising of wavelengths that water reacts to

A ratio of I_{filt1} and I_{filt2} may be determined as exemplified by EQ4.

$$Ratio = \frac{{I}_{filt 1}}{{I}_{filt 2}} = \frac{{I}_{0} 1 {0}^{- {μ}_{filt 1} c l}}{{I}_{0} 1 {0}^{- {μ}_{flit 2} c l}} = {10}^{( - {μ}_{filt 1} cl - ( - {μ}_{filt 2} cl ) )} \qquad EQ4$$

A ratio of I_{filt1} and I_{filt2} may be determined as exemplified by EQ4 and EQ5.

$$Ratio = \frac{{I}_{filt 1}}{{I}_{filt 2}} = {10}^{- c l ( {μ}_{filt 1} - {μ}_{filt 2} )} \qquad EQ5$$

Since the optical path length and absorption coefficients are fixed, factor x corresponds to a constant, as exemplified by EQ6.

$$x = l ( {μ}_{filt 1} - {μ}_{filt 2} ) \qquad EQ6$$

It follows that the ratio is directly linked to moisture c, as x is a constant, so the only parameter that may change the result is the water concentration, as exemplified by EQ7.

$$Ratio = \frac{{I}_{filt 1}}{{I}_{filt 2}} = {10}^{- c x} \qquad EQ7$$

It follows that when the sensor element is adapted to individually acquire different light intensities, each associated with a defined individual wavelength or wavelength band, the water concentration, and thus the moisture content of the object, e.g., hair, may be determined by building the ratio of the individual measurements. The resulting value is thus indicative of a certain moisture content.

In addition to the measurement signal, when determining the moisture content, additional parameters or factors may be taken account like a temperature of the hair, a distance between the haircare appliance and the hair, a hair presence, an environmental condition, an ambient temperature, an ambient air pressure, an ambient light level, an ambient humidity, a hair colour, a hair melanin content, and a user input. In particular, a user input may be beneficial, as such user input may already provide a rough indication of the measurement circumstances that currently apply. For example, the user input may be specifying a general hair colour range, e.g., light or dark hair, which may directly influence the moisture content determination by adapting the measurement procedure. Other user input is likewise conceivable, e.g., specifying a current ambient temperature, the lighting conditions of the area where the haircare appliance is currently used, a hair type, a hair length, a current hair style and/or a target hair style, in order to adapt the acquisition of the sensor signal.

The user input may be directly set at the haircare appliance, or a separate device, remote, or app in communicative connection with the haircare appliance may be used. For example, the user may have a dedicated user profile in a smartphone application, so that potentially a plurality of parameters specific to the user may be set and provided to the haircare appliance in a quick and easy manner before starting a styling operation. The application may have added functionality for specifying user parameters. For example, the user may take a picture, with the app subsequently analysing the picture to determine the hair colour, hair structure and further relevant properties influencing the moisture content determination. The analysis may be performed using AI technology.

The system may operate in one of two modes where either absolute or relative measurements are obtained. In case of absolute measurement, hair moisture result will be provided at any given time from the area of exposure or acquisition regions. In a relative mode, the calibration procedure may be conducted at stable and dry hair condition to provide reference dry hair data. Alternatively, a relative change of moisture may be monitored.

Sensor readout may in particular be dependent on hair colour. For a beneficial moisture content determination, the sensor may be calibrated for variations in hair colour. Additionally, or alternatively to a user input, the haircare appliance may comprise an additional hair colour detection sensor (e.g., an RGB sensor or camera) that provides colour information. The dedicated hair colour detection sensor element may use a spectral sensitivity band (e.g., 700 to 1700 nm) identical or similar to the spectral sensitivity of the sensor element. In between moisture measurement time slots, an additional LED may emit pulsed light at a wavelength that is correlated to hair colour. The response is captured by a, in particular unfiltered, photodiode and the information may subsequently be normalised by a filtered photodiode present in the sensor element (e.g., at about 1450 nm). Alternatively, the measurement may be conducted in time division multiplex by capturing sensor data in different time slots with a single light source and detector, where in particular different filtering is used for different timeslots.

The sensor element may be self-calibrating during normal use through analysing measurements taken at a defined point in a styling procedure such as after wrapping a section of hair when it is known that the hair is dry. The haircare appliance may further indicate to the user to take a calibration measurement. It is also conceivable that a particular haircare appliance is specifically pre-calibrated to a defined hair colour, i.e., the haircare appliance may be calibrated out of the box by a prior calibration procedure.

Still further, it is conceivable that more hair colour compensation may be necessary by using a specific spectral band for water detection (e.g., at about 1920 nm).

The haircare appliance may further comprise hair presence detection, e.g., the detection of the presence of a hair tress in the region of interest/an acquisition region using the correlation of the signal from two channels of the sensor element. Such a procedure may extend the dynamic range below the level detectable by the ratio of the two channels. In other words, if hair is not present, the output of the sensor element fall below a given level. By applying an appropriate threshold, it may be determined if/when a target is in acquisition range of the sensor element and the sensor reading may be analysed whether is corresponds to an expected measurement value.

Erroneous data may thus be removed from a calculation if the sensor element output falls outside of given ranges. A correlation function may perform analysis of the measurement signals from the sensor element to determine if they are following a similar trend over time i.e., looking at the same target. If the photodetector signals are not looking at the same target, the signals are random and there is no correlation between sensor signals in a comparably short period of time (i.e., windowing is performed).

The haircare appliance, in particular the accessory, may be adapted to position the hair relative to the sensor element, e.g., by a guiding element or channel, so that the hair is presented to the sensor in a defined way, e.g., at a defined angle and/or distance, to optimize the acquisition of the sensor reading. Further, the haircare appliance may comprise a camera element for determining a position of the hair relative to the sensor element. Knowing, setting or determining the relative alignment of the hair and the sensor element, may assist in acquiring a meaningful sensor reading, or may allow the post-processing of the sensor reading to take into account the relative alignment of the hair and the sensor element.

The sensor element may be part of a separate device, e.g., a brush. In other words, the sensor element may be integrated in a device separate from the main body of the haircare appliance and arranged to wirelessly transmit moisture data using a wireless communication connection, e.g., Bluetooth, NFC, RFID or WLAN to the main body of the product.

According to an embodiment of the present disclosure, the element for obtaining at least one sensor reading may be an element for wirelessly transmitting the information related to the sensor reading from the acquisition region to the sensor element.

According to a further embodiment of the present disclosure, the element for obtaining at least one sensor reading may be the sensor element.

Wirelessly in the context of the present disclosure may be any transmission modality that is not specifically wired. For example, the element may be an element for sending electromagnetic radiation, radiofrequency radiation and more particularly light in the visible range, infrared range, or ultraviolet range. The element for obtaining at least one sensor reading may itself transmit or propagate the information, e.g., may be a light propagating element like a light pipe or may simply wirelessly send the information, e.g., by using radiofrequency radiation between a transmitter and a receiver. In this scenario, the element for obtaining at least one sensor reading may be passively transmitting the information in the case of a light propagating element or may be actively transmitting the information in case of a radiofrequency transmission scenario.

Alternatively, the element itself may be the sensor element, thereby directly acquiring a sensor reading or a measurement signal. Still further, the element for obtaining at least one sensor reading may be a combination of the aforementioned, e.g., may be a sensor element with an associated or attached light propagating element.

According to a further embodiment of the present disclosure, the sensor element may be arranged in the main body, or the sensor element may be arranged in the accessory, or the sensor element may be arranged in an intermediary element arranged between the main body and the accessory. In particular, the sensor element may be arranged in the main body, the haircare appliance may further comprise an intermediary element arranged between the main body and the accessory, and the intermediary element may comprise a further element for wirelessly transmitting the information related to the sensor reading, in particularly for transmitting information related to the sensor reading and/or a sensor reading initiation signal between the main body and the accessory.

Employing an element for obtaining at least one sensor reading which is in particular wirelessly transmitting the information related to the sensor reading, i.e., transmitting a sensor signal from the sensor element to the acquisition region and a measurement signal from the acquisition region to the sensor element, allows the flexible placement of the sensor element in the interior of the haircare appliance. By using a wireless transmission, it may not be necessary to place a sensor element in the vicinity of the acquisition region since by using the wireless transmission, the sensor element may not need to directly interact with the acquisition region or directly acquire measurement information from the acquisition region but can acquire such via the element for obtaining the sensor reading.

According to a further embodiment of the present disclosure, the sensor element may be functionally associated with the element for obtaining at least one sensor reading, so that the information related to the sensor reading transmitted from the acquisition region is receivable by the sensor element.

In other words, signals emitted from the sensor element may be propagated by the element for obtaining at least one sensor reading towards the acquisition reading while a measurement signal may be propagated by the element for obtaining at least one sensor reading to the sensor element. In case of an optical transmission, the sensor element is functionally associated with the element for obtaining at least one sensor reading when there is a working transmission between the sensor element and the element for obtaining at least one sensor reading. For example, in a case where the element for obtaining at least one sensor reading is arranged in close spatial proximity to signal emitter and/or signal receiver elements of the sensor element, a functional association may be achieved. In a case where the sensor element is only receiving sensor information, the sensor element may be considered a passive sensor element. Receiving only information may be sufficient to assume the functional association of the sensor element with the element for obtaining at least one sensor reading.

According to a further embodiment of the present disclosure, the sensor element may be an active sensor element for actively generating a sensor reading initiation signal that is transmitted to the accessory to initiate the information related to the sensor reading.

According to a further embodiment of the present disclosure, the sensor reading initiation signal may be transmitted to the acquisition region through the element for obtaining at least one sensor reading, and/or the sensor reading initiation signal may be transmitted to the acquisition region through a further transmission element.

Thus, the sensor element in turn may initiate a sensor signal to be transmitted to the acquisition region. In other words, the sensor element may generate at least one particular electromagnetic radiation of a defined frequency or frequency band, which are propagated to the acquisition region. The electromagnetic radiation may subsequently interact with an object in the acquisition region, thereby generating a measurement signal, which in turn may be propagated back to the sensor element as a measurement response to the sensor signal/the sensor reading initiation signal. Depending on the type of target, it may be conceivable that the electromagnetic radiation at least partly passes through the target when obtaining a measurement, rather than being reflected off the target. The target may thus be arranged between the source of the electromagnetic radiation and the sensor element.

An active sensor element may be an element adapted for actively sending a sensor signal to initiate a reaction that in turn is acquired by the sensor element as a measurement signal. It is further conceivable that the sensor element comprises an active sensor element part for sending the sensor signal as well as a passive sensor element part for receiving the measurement signal. The at least one active sensor element part and the at least one passive sensor element part may constitute a single sensor element or may be separate elements, in particular spatially separated, functionally independent electronic elements. In the case of electromagnetic radiation in the range of light, the sensor reading initiation signal may be generated by a light emitting diode having the appropriate frequency or frequency range. Filter elements may be used to adapt a generated frequency spectrum from a LED to a desired frequency or frequency range.

Initiating the information related to the sensor reading may in particular be understood as generating a reaction from the sensor signal by an object in the acquisition region. The sensor reading initiation signal may also be referred to as sensor signal, i.e., a signal that is emitted by the sensor element, wherein the sensor reading may also be referred to as measurement signal, i.e., a signal that is received by the sensor element.

According to a further embodiment of the present disclosure, the haircare appliance may comprise a plurality of acquisition regions and/or a plurality of sensor elements, the haircare appliance may comprise a plurality of elements for obtaining at least one sensor reading and/or a plurality of further transmission elements, and the plurality of elements for obtaining at least one sensor reading and/or the plurality of further transmission elements may connect the plurality of acquisition regions and/or the plurality of sensor elements.

In addition to having a single acquisition region, the haircare appliance may be arranged to acquire measurement information of a plurality of sensor regions. The measurement information may be propagated to a single sensor element, or a plurality of sensor elements may be provided which are individually associated with a particular acquisition region. By appropriately embodying the elements for obtaining at least one sensor reading or further transmission elements, the at least one sensor element may be appropriately placed away from the acquisition region, potentially arranged within the interior of the haircare appliance.

According to a further embodiment of the present disclosure, the sensor element may be adapted to generate the sensor reading initiation signal, the sensor reading initiation signal may be transmitted to the acquisition region, the sensor reading initiation signal may interact with an object at or around the acquisition region for generating the information related to the sensor reading, the element for obtaining at least one sensor reading may be adapted to transmit the generated information related to the sensor reading to the sensor element, the sensor element may be adapted to receive the information related to the sensor reading, and the sensor element may be adapted to process the received information related to the sensor reading to obtain the sensor reading.

In other words, the haircare appliance comprises an active sensing system where a substantially freely placed sensor element generates a sensor signal which is transmitted to the acquisition region. In reaction to the sensor signal, the object arranged within the acquisition region interacts with the sensor signal and, in turn, a measurement signal, a measurement response signal or measurement response information is generated, which again is propagated back to the sensor element.

According to a further embodiment of the present disclosure, the sensor reading may be a sensor reading of an attribute of hair treatment currently treated by the haircare appliance, and the attribute may be indicative of at least one parameter out of the group consisting of a moisture level of the hair, a hair moisture, temperature of the hair, a distance between the haircare appliance and the hair, a distance between the accessory and the hair, a hair presence, an environmental condition, an ambient temperature, an ambient air pressure, an ambient light level, an ambient humidity, a hair colour, a hair melanin content, and a user input.

According to a further embodiment of the present disclosure, the sensor element may be arranged in the haircare appliance so to be not exposed to the physical operation of the blower, the airflow of the blower and/or heat provided by a heating element for heating the airflow.

In other words, the sensor element may be arranged so to be out of reach of the operation of the blower or the heater and thereby be protected. In particular, in case the sensor element is sensitive to a change in ambient temperature which may result in a reduced measuring accuracy, it may be beneficial to arrange the sensor element in a part of the haircare appliance that is not affected by the operation of the heater. Thereby, the temperature surrounding the sensor element may be kept in a defined range and thereby the measurement accuracy may increase.

According to a further embodiment of the present disclosure, the processing element may be adapted to employ the at least one parameter to determine a compensation factor for the sensor reading when determining the moisture content.

Many factors or parameters influence acquisition of a sensor signal and may thus lead to an inaccurate moisture content determination. It may thus be beneficial to consider these factors or parameters when determining a moisture content. In other words, for a sensor element acquiring a sensor signal in order to determine the moisture content of hair, the sensor signal itself may not only be influenced by the moisture content itself but also by peripheral factors such as colour of the hair, a current ambient temperature, a current ambient humidity level, or simply the presence or rather the lack of hair in the vicinity of the sensor element or an acquisition region. Likewise, the amount of hair may influence a sufficiently reliable moisture content determination, as a case where only a comparably small amount of hair is arranged in the vicinity of the sensor element or within the acquisition region may lead to a different determinant moisture content compared to a situation where various the significant amount of hair in the vicinity of the sensor element or within an acquisition region. By including such factors, variables or parameters when determining the moisture content, the reliability and accuracy of a determined moisture content value may be increased.

A temperature sensor may also be placed near the spectral sensor or may be integrated within the sensor element to compensate for temperature changes or a specific temperature at the time of taking a measurement. Alternatively, a temperature change may be estimated from sensor output data when the sensor element is not measuring hair.

According to a further embodiment of the present disclosure, the haircare appliance may comprise a single sensor element adapted to receive a plurality of spectral bands, and the single sensor element may have at least two sensor sections, wherein the at least two sensor sections may be adapted to receive different spectral bands, and/or the single sensor element may employ a plurality of filter elements or a dynamic filter element having a plurality of filter characteristics, to receive the different spectral bands, or the haircare appliance may comprise at least two sensor elements, where each sensor element may be adapted to receive a respective spectral band, wherein the respective spectral bands are different.

According to a further embodiment of the present disclosure, the sensor element may be a spectral sensor element, wherein the sensor element may in particular be operated in a time-division multiplexing mode.

By determining a spectral response in two different, in particular independent, partially independent or unrelated spectral bands, a moisture content value may be determined with increased accuracy and reliability. For example, one spectral band may relate to a part of the spectrum that is particularly influenced, e.g., absorbed, by moisture in the hair whereas a further spectral band may relate to a part of the spectrum that is particularly unaffected or only affected to a reduced extent by the moisture content in the hair. By comparing measurements related to the respective spectral bands, in particular by establishing a ratio between the measurements, detrimental effects that result from variables, factors or parameters other than the moisture content that result in a reduced accuracy or reliability of moisture content determination may be compensated for. For example, measuring in a spectral band which is largely unaffected by the moisture content while at the same time largely affected by the hair colour, i.e. the melanin content, and comparing the measurements with a measurement in a further spectral band which is largely affected by the moisture content while at the same time largely unaffected by the hair colour may allow the determination of the hair colour/melanin content on the one hand while at the same time using said information to determine the moisture content with high accuracy and reliability.

Using a sensor element in a time-division multiplexing mode may allow to obtain a plurality of measurements where part of the measurements is affected by the moisture content whereas another part is unaffected by the moisture content, or alternatively may allow to obtain a plurality of measurements where part of the measurements is affected by one of the aforementioned factors, parameters or variables while another part is unaffected, in order to compensate for any effect that would be detrimental to the reliable and accurate moisture content determination. For example, in the case of a spectral sensor, a sensor element in a time division multiplexing mode may determine a measurement in a first spectral band during a first time period while determining a measurement in a second spectral band during a second time period. Also, in time division multiplexing, an EM radiation source may be disengaged/switched off in a defined time period so that the sensor element may acquire an ambient light level to further compensate any effect on the measurement of the ambient light. Such an arrangement would result in the availability of two sets of measurements which again can be used for parameter compensation.

According to a further embodiment of the present disclosure, the haircare appliance may further comprise at least one of a time-of-flight sensor element, a proximity sensor element, a distance sensor element, a contact sensor element, a light intensity sensor element, a microwave sensor element, and a capacitive sensor element to determine the hair presence and/or the distance between the haircare appliance and the hair, and/or the sensor element may be adapted for a time-of-flight measurement to determine the hair presence and/or the distance between the haircare appliance and the hair.

A time-of-flight sensor may, for example, measure the time it takes an emitted signal to return to the sensor, e.g., by reflection. Such an emitted signal may exemplary be a visual signal, a haptic signal or an acoustic signal. Preferably, such an emitted signal may have a pulse like structure, in particular having a sharp rising or falling edge in order to accurately determine the travel time. By using such a time-of-flight sensor, the distance between the sensor element and the object responsible for the reflection may be determined with high accuracy. The travel time of a signal will be comparably small in a case where hair is present in the vicinity of the sensor element whereas the travel time will be larger in a case where hair is further away from the sensor element or substantially not present in the vicinity of the sensor element, i.e., not present within the sensing range of the sensor element. By continuously monitoring a time-of-flight sensor value, the moving away of hair from the haircare appliance or the moving closer of hair to the haircare appliance can be determined with high reliability. A time-of-flight measurement may then be correlated and/or fused with other measurements in order to exclude measurements where it can be determined that hair was not in the proximity of the sensor, in other words sufficiently close to constitute a reliable measurement.

According to a further embodiment of the present disclosure, the haircare appliance may further comprise at least one temperature sensor, wherein the at least one temperature sensor may be arranged in the vicinity of the sensor element, and/or wherein the at least one temperature sensor may be arranged in the vicinity of the acquisition region.

According to a further embodiment of the present disclosure, the at least one temperature sensor may be acquiring a temperature value in the vicinity of the sensor element and/or in the vicinity of the acquisition region, and wherein the powering of the blower, the powering of a heater, and/or the powering of a cooling element for cooling the vicinity of the sensor element and/or the vicinity of the acquisition region may be dependent on the acquired temperature value.

By providing a temperature sensor in the vicinity of the sensor element and/or the acquisition region, both an ambient temperature and/or a temperature resulting from the operation of the haircare appliance may be determined with high reliability. Since such a temperature may influence measurements performed by the sensor elements, using the information on said temperature may result in an increase in reliability and accuracy of a moisture determination. For example, measurement values may be compensated for the influence of a particular temperature on the measurement value. Additionally, or alternatively, the temperature value may be used for controlling the operation of the hair care appliance. Still further, the determined temperature value may be used to influence the blower, heater, or a further cooling element in order to reduce the temperature in the vicinity of the sensor element and/or the acquisition region. For example, in a case where a temperature is determined which would result in an unreliable moisture determination, a power of the blower may be increased, a power to a heater element may be reduced and/or a cooling element for cooling the sensor element and or the acquisition region may be activated, potentially resulting in a temperature reduction at or around the measurement region.

According to a further embodiment of the present disclosure, the haircare appliance may further comprise a colour sensor element to determine the hair colour and/or the melanin content of the hair to be treated, and/or the sensor element may further be adapted for a hair colour measurement to determine the hair colour and/or the melanin content of the hair to be treated.

According to a further embodiment of the present disclosure, the haircare appliance is adapted to receive a user input to specify the hair colour and/or the melanin content of the hair to be treated.

By specifically determining the colour of the hair and/or the melanin content, said value may be used when determining the moisture content of the hair. Since a sensor reading, in particular a spectral sensor reading, for determining the moisture content may be particularly dependent on the melanin content or the colour of the hair, by knowing said parameters, the effect of different hair colour may be taken into account and appropriately compensated when taking a measurement and/or when calculating a moisture content from the measurement.

In addition to obtaining the hair colour or melanin content by a dedicated measurement, a user may also specify a hair colour value and/or a value related to melanin content of the hair to be treated. Such a user input may not necessarily be as exact as a dedicated hair colour or melanin content sensor measurement, however a rough information about a hair colour may generally be sufficient to allow an adaptation or compensation of a moisture measurement when determining the effective moisture content of the care. For example, specifying whether the hair is light coloured, e.g., blonde, or dark coloured, e.g., brown or black, may allow an initial adaptation or compensation of a measurement value when determining the moisture content. The user input may be a manual input where the user selects a specific hair colour out of a range of hair colours, or may be an automatic input, e.g., by the user taking a photo of the hair. Still further, it is conceivable that the haircare appliance recognises a specific user currently using the haircare appliance while having further stored information on the hair colour of said user. With this information, the haircare appliance, when determining the moisture content of the hair, may utilise the hair colour or melanin content information.

Still more preferably, it is conceivable that a user input regarding a specific hair colour is complemented with a dedicated hair colour or melanin content measurement. When first manually specifying the hair colour, a hair colour determination may be conducted in a preferred measurement range based on the prior rough knowledge of the hair colour expected to be measured.

According to a further embodiment of the present disclosure, the sensor element may be arranged spaced apart from the acquisition region, information related to the sensor reading may be transmitted from the acquisition region to the sensor element by an element for obtaining at least one sensor reading, and the information may be transmitted wirelessly, in particular transmitted optically.

Having a sensor element that is arranged spaced apart from the acquisition region allows to protect the sensor element in that it is not exposed in a region that may be constant contact with the hair of a user. In particular, in a case where the sensor element is arranged in the interior of the haircare appliance and thus it is not possible to directly contact the sensor element, such would result in a reduced possibility of damaging the sensor element by accident. Also, in case the haircare appliance is dropped, or an unusual force is otherwise acting on the haircare appliance, arranging the sensor element away from a possible external access reduces the risk of permanent damage to the sensor and thus the haircare appliance. Transmitting information related to the sensor reading from the acquisition region to the sensor, in particular wireless transmission, allows the placing of the sensor with a high degree of design freedom.

According to a further embodiment of the present disclosure, the haircare appliance may comprise a main body comprising a blower for generating an airflow and the sensor element, wherein the main body may comprise an attachment region adapted for connecting an accessory, and an accessory wherein the accessory may be attachable to and/or detachable from the main body at the attachment region, wherein the accessory, when attached to the main body, may be adapted to receive the airflow from the blower, and wherein the accessory may be adapted to discharge the received airflow towards the hair of a user, wherein radiation from the light emitting element may be transmitted to the acquisition region and the information related to the sensor reading may be transmitted from the acquisition region to the sensor element by the element for obtaining at least one sensor reading.

Arranging the sensor element in the main body of a haircare appliance means propagating measurement information and/or measurement signals between the sensor element and the acquisition region allows the provision of a single sensor element while using the measurement functionality with a plurality of different accessories. Such a central sensor element may thus allow to design a haircare appliance more economically, while still providing the measurement functionality for each or at least select accessories. Further, arranging the sensor element in the interior of the main body allows to protect the sensor element without a negative impact on the measurement quality. By appropriately adapting the element for obtaining at least one sensor reading to each accessory allows providing optimal measurement results without the requirement to find a single placement solution for all accessories.

When the sensor element is positioned in a main body of the haircare appliance, it may work with multiple different attachments. The sensor element may require information which attachment is currently attached/in use in order to calibrate measurements. Each attachment may have a unique optical property so that the sensor element may automatically recognise the attachment based on the back-reflected signal and adapt the measurement and/or operation of the haircare appliance accordingly.

According to a further embodiment of the present disclosure, the haircare appliance may comprise a plurality of acquisition regions and/or a plurality of sensor elements, the haircare appliance may comprise a plurality of elements for obtaining at least one sensor reading and/or a plurality of further transmission elements, and the plurality of elements for obtaining at least one sensor reading and/or the plurality of further transmission elements may connect the plurality of acquisition regions and/or a plurality of sensor elements.

The elements for obtaining at least one sensor reading or the further transmission elements thus allow the flexible connection of sensor elements to acquisition regions. For example, a single sensor element may be connected to a plurality of acquisition regions in order to spread out the individual points of acquisition, effectively resulting in an increase of the acquisition region. This in turn allows the acquisition of a plurality of measurements substantially in parallel, without the need for providing multiple sensor elements. Alternatively, or additionally, providing a plurality of sensor elements allows the use of possibly different sensor elements or sensor types while acquiring measurement information at or around substantially the same acquisition region. Using elements for obtaining at least one sensor reading or the further transmission elements thus provides added flexibility when designing the measurement functionality for acquiring sensor readings to determine a moisture content of the hair. For example, in the case of an elongated accessory, it is conceivable to have different acquisition regions spread along the length and/or around the circumference of the accessory, thereby increasing the effective size of the acquisition region without the need to provide a plurality of sensor elements.

According to a further embodiment of the present disclosure, the element for obtaining at least one sensor reading may be a light propagating element, and the light propagating element may be arranged for transmitting the information related to the sensor reading as electromagnetic radiation from the acquisition region to the sensor element.

According to a further embodiment of the present disclosure, the element for obtaining at least one sensor reading may be at least one element or arrangement out of the group consisting of an optical element for collecting, transporting/guiding and distributing light, a lens, a mirror, a hollow prismatic pipe, a light rod, a mirrored light pipe, an optical fibre and an optical filter.

By using electromagnetic radiation, measurement information may easily be guided from the acquisition region to the sensor element. At the same time, it is conceivable that a further signal is guided from the sensor element to the acquisition region. For example, in a case where the measurement is conducted using light, visible light, ultraviolet radiation or infrared radiation, signals required for taking a measurement may be propagated from the sensor element arranged away from the acquisition region to the acquisition region, while at the same time measurement information may be propagated using the same or a different element from the acquisition region to the sensor element. For example, in a case of an active sensor, electromagnetic radiation required for taking the measurement may be propagated from the sensor element to the acquisition region, while the measurement information, e.g., an altered or reflected part of the electrical radiation, may be propagated back to the sensor element. Preferably, the element for obtaining at least one sensor reading may be a flexible and, in particular, heat insensitive element, so that it can be arranged easily in the interior of the haircare appliance. Using a sensor element attached to such optical elements, the acquisition of the hair a sensor element may be performed in an extended range, i.e., not only specifically in the vicinity of a surface of the haircare appliance or the accessory but even in a certain distance from the end of the element for obtaining at least one sensor reading. For example, in a case where the sensor element, by using additional optical elements, has a homogeneous field of view, even at a distance from the end of the element for obtaining at least one sensor reading, it may not be necessary that the hair is particularly close to the surface of the haircare appliance or the accessory.

According to a further embodiment of the present disclosure, the operation of the haircare appliance may be adaptable dependent on the sensor reading and/or the determined moisture content, further in particular dependent on the type of accessory attached to the main body.

According to a further embodiment of the present disclosure, the powering of the blower, the powering of a heater associated with the blower and/or the powering of the accessory may be dependent on the sensor reading and/or the determined moisture content.

According to a further embodiment of the present disclosure, the haircare appliance may be adapted to provide a visual, haptic and/or acoustic signal to the user, wherein the visual, haptic and/or acoustic signal may be dependent on at least one of a mode of operation of the haircare appliance, an accessory type attached to the haircare appliance, a moisture content, and a hair colour.

In other words, the sensor reading may directly influence how the haircare appliance is operating. For example, in a case where a certain sensor reading like a moisture content is determined to be at a level where it would be detrimental to the health of the hair or to the styling experience, the haircare appliance may be switched off or may be put in an alternative configuration such as reducing the airflow or the temperature. Alternatively, or additionally, the haircare appliance may signal a user that a certain styling condition, e.g., a desired remaining moisture content is met, so that the user in turn may react appropriately, e.g., by terminating the styling. Still further, it is conceivable that the behaviour of the haircare appliance changes depending on the type of accessory. For example, in a case where the accessory, due to its construction, requires only a reduced airflow, the operation of the haircare appliance may be adapted so that a maximum airflow cannot be exceeded, e.g., despite a user trying to set such airflow. Likewise, in case the maximum styling temperature for a particular accessory should not exceed a certain threshold below the maximum styling temperature, the haircare appliance may regulate the temperature such that said maximum styling temperature is not exceeded.

### BRIEF DESCRIPTION OF THE DRAWINGS

The present invention will now be described with reference to the accompanying drawings, in which:

FIG. 1 shows an exemplary embodiment of a haircare appliance according to the present disclosure.

FIGS. 2A and 2B show schematic diagrams of haircare appliances according to the present disclosure.

FIG. 3 shows exemplary embodiments of a sensor element according to the present disclosure.

FIG. 4 shows a schematic diagram of haircare appliance according to the present disclosure.

FIGS. 5A and 5B show cross-sectional views of exemplary embodiments of haircare appliances according to the present disclosure.

FIGS. 6A to 6M show cross-sectional views of exemplary embodiments of haircare appliances according to the present disclosure.

FIG. 7 shows cross-sectional views of an exemplary embodiment of a haircare appliance according to the present disclosure.

FIGS. 8A to 8D show views of exemplary embodiments of haircare appliances according to the present disclosure.

FIGS. 9A to 9C show exemplary embodiments of measurement compensation according to the present disclosure

### DETAILED DESCRIPTION

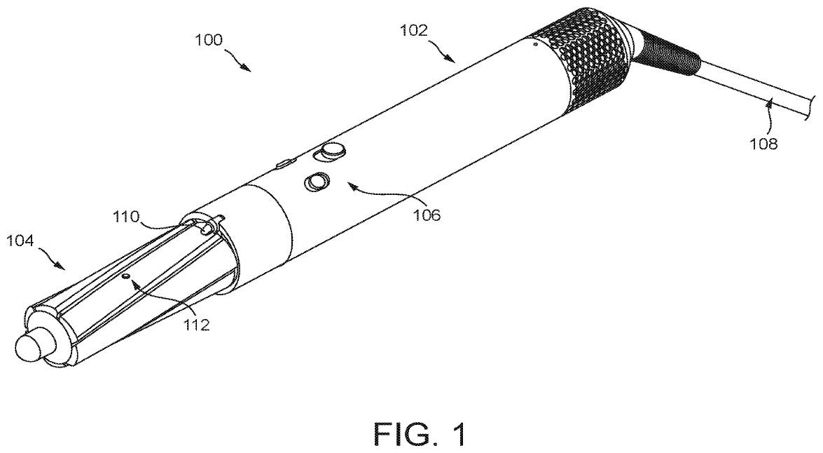

Now referring to FIG. 1, which shows an exemplary embodiment of a haircare appliance according to the present disclosure.

Haircare appliance 100 in FIG. 1 is exemplarily a hair curler, comprising a main body 102 and an accessory 104. Haircare appliance 100 is a cord powered appliance with power cord 108 connecting to a wall outlet. The haircare appliance 100 comprises a user interface 106 or knobs/switches for controlling the operation of the haircare appliance 100. For example, a switch may generally activate or deactivate operation of the haircare appliance 100, while further knobs may set a turning direction and/or selectively activate/deactivate a motor, a blower and/or heater.

The haircare appliance 100 comprises a sensor element in its interior, in particular in the main body 102, which is however not depicted in FIG. 1. In order to obtain a measurement from hair arranged adjacent to the accessory 104 when using the haircare appliance 100, the accessory 104 has an opening 112 so that a measurement signal may be propagated to the sensor element by using the element for obtaining a sensor reading 110, exemplarily a light in FIG. 1. This in other words, when hair is arranged around the circumference of the accessory 104, a sensor reading may be taken through a window 112. In order to obtain a sensor reading, the sensor element itself may emit at least one signal, e.g., radiation having a specific wavelength. Said signal or signals may then be propagated through the element for obtaining the sensor reading 110 to the hair, in particular be emitted onto the hair through opening 112. The signal or signals may then interact with the hair and in turn provide reactive signals, which are then propagated back through the element for obtaining the sensor reading 110.

The sensor element itself may be a passive sensor element, merely receiving an externally generated sensor signal, or may be an active sensor element, itself emitting a sensor signal which in turn is reflected by the hair arranged at the accessory 104. Depending on the measurements, and operation of the haircare appliance 100 may be automatically set, irrespective of a user input via the user interface 106.

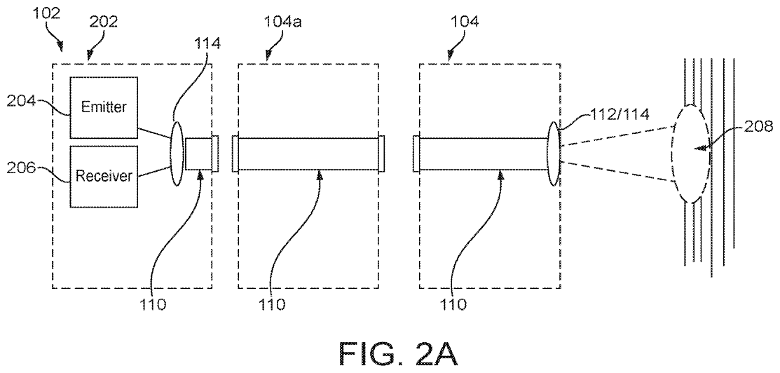

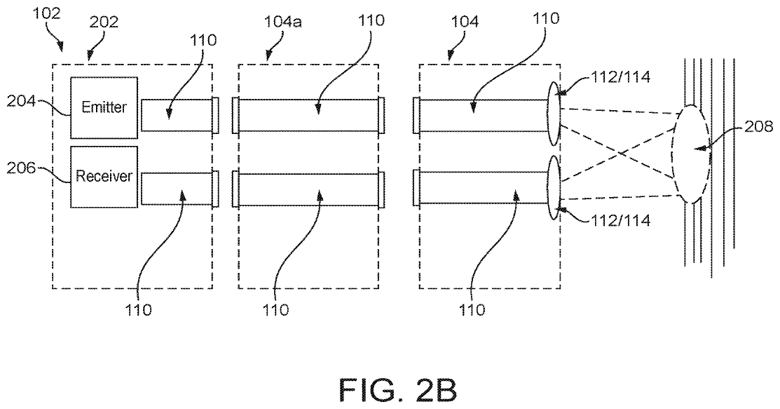

Now referring to FIGS. 2A and 2B, which show schematic diagrams of haircare appliances according to the present disclosure.

FIGS. 2A, B schematically show how the sensor element 202 is operating internally of the haircare appliance 100. Sensor element 202 is exemplarily arranged in the main body 102 of the haircare appliance 100, and itself comprises an emitter 204 and a receiver 206. The emitter 204 may e.g., be a light emitting diode, emitting electromagnetic radiation having a visible, an infrared or ultraviolet wavelength. Arranged adjacent to the emitter 204 and the receiver 206 is optical element 114, e.g., a lens for focusing the emitted sensor signal and introducing the sensor signal into the element for obtaining a sensor reading/light pipe 110. The emitted signal is thus propagated through light pipe 110 and is emitted through opening 112 possibly also comprising an optical element 114, e.g., a lens, onto an object 208 or hair.

The emitted signal in turn is reflected back from the hair 208 and is propagated back through light pipe 110 towards the receiver 206. Receiver 206 receives the reflected signal and in turn is adapted to analyse the signal in order to generate a measurement signal, which e.g., may be used to determine the moisture content of the object 208 in the vicinity of opening 112. Light pipe 110 consists of three parts, wherein a first part is arranged in the main body 102, while a second part is arranged in attachment 104. Optionally, the haircare appliance 100 may comprise a further intermediate accessory or attachment element 104a, situated between the main body 102 and the accessory 104. In the embodiment of FIG. 2A, the signal emitted from emitter 204 thus shares the same light pipe 110 with the reflected signal propagating towards the receiver 206.

An alternative solution is shown in FIG. 2B, where the main difference is that the emitter 204 and the receiver 206 each have a dedicated, separate light pipe 110, one light pipe 110 for propagating the signal towards the hair 208, while a different light pipe 110 is propagating the signal reflected from the hair 208 back to the receiver 206. In this scenario, it may be conceivable to omit the optical element 114 in the main body 102, and directly couple the emitted signal into or out of the light pipe to and from the respective emitter 204 and receiver 206 elements.

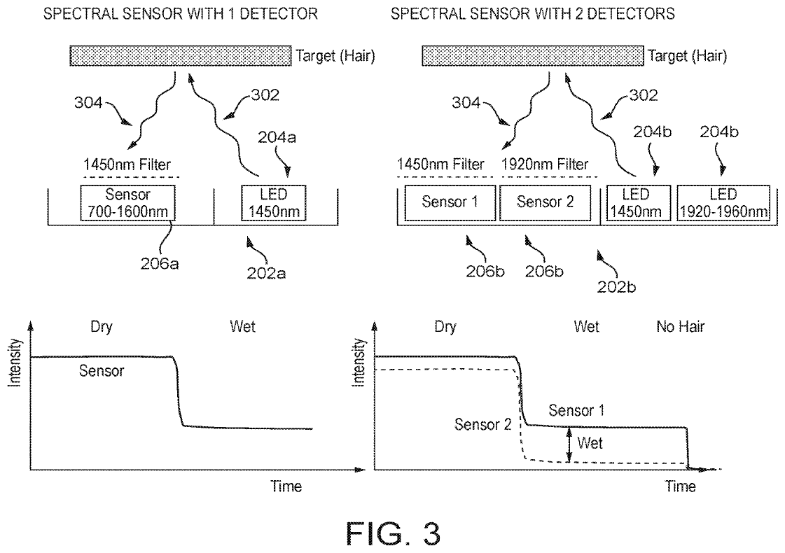

Now referring to FIG. 3, which shows exemplary embodiments of a sensor element according to the present disclosure.

In FIG. 3, two embodiments of sensor elements are shown, one sensor element 202a or spectral sensor (left side) comprising a single detector or sensor section as well as one sensor element 202b or spectral sensor (right side) comprising exemplarily two detectors or sensor sections. FIG. 3 also shows the measurement response of intensity vs. time of the respective sensor element of dry and wet hair.

Sensor element 202a comprises an emitter 204a emitting a sensor signal 302 towards object 208. The emitting and receiving of sensor signals in FIG. 3 are only depicted schematically, in particular depicted without any element for obtaining a sensor reading 110/light pipe. The emitted sensor signal 302 is emitted exemplarily from a light emitting diode LED having a defined wavelength, e.g., 1450 nm. The emitted sensor signal 302 is reflected from target 204 towards the single detector or sensor section 206a. A filter element allowing the passage of a wavelength of exemplarily 1450 nm is shown. It is conceivable that in case the emitter 204a is an emitter having a defined wavelength, the filter element is not required. Alternatively, the emitter 204a may be implemented as a broadband emitter, in which case the filter element is preferably provided. The received sensor signal 304, reflected of object 204, passes through the filter element, in case it is provided, and arrives at the receiver 206a. The receiver itself may be a broadband receiver, e.g., for receiving light of a wavelength between 700 and 1600 nm.

The diagram below sensor element 202a shows the measurement response of the sensor. For a defined emission intensity, the received intensity for dry hair is higher than for wet hair. In other words, wet hair is more absorbent than dry hair. Without knowledge of the specific emission intensity, the determination of whether the hair is wet or dry may not be conclusive, as the received intensity is dependent on a plurality of factors, and the moisture content of the hair is only one of said parameters

Sensor element 202b exemplarily comprises two emitters 204b and two receivers 206b, e.g., two detectors or sensor sections. Each of the emitters 206b emits a sensor signal 302 of a defined wavelength or wavelength band, exemplarily 1450 nm and 1920 to 1960 nm. The emitters 204b may operate simultaneously, or in a timed sequence with only one of the emitters being active at any one time. The emitted sensor signal 302 is reflected from target 204 towards the exemplarily two receivers 206b. Two filter elements are provided with their respective filter pass wavelengths adapted to the emitted wavelengths of the emitters 204b. In case that filter elements are provided, the emitters 204b may be broadband emitters or may even be a single emitter since the separation of the emitted electromagnetic radiation is performed by the filter elements before the radiation reaches the receivers 206b. In case of wavelength selective emitters as depicted in FIG. 3, that are operated in a timed sequence, it is conceivable that only a single receiver 206b is provided, that is used for the intensity measurements in a time multiplex manner.

In FIG. 3, the receivers 206b receive the sensor signals 304 substantially simultaneously. As can be taken from the diagram associated with sensor element 202b, a difference in intensities in dry hair between sensor 1 and sensor 2, i.e., between the different wavelengths, is smaller than when the hair is wet. By using the Beer Lambert Law as described previously, by calculating a ratio of the respective intensities, it can be determined whether the hair is wet or dry.

Still further, the right diagram exemplifies the detection that no hair is present. Since no emitted sensor signal is reflected back from the target to the receivers, the measured intensities are comparably small and essentially equal.

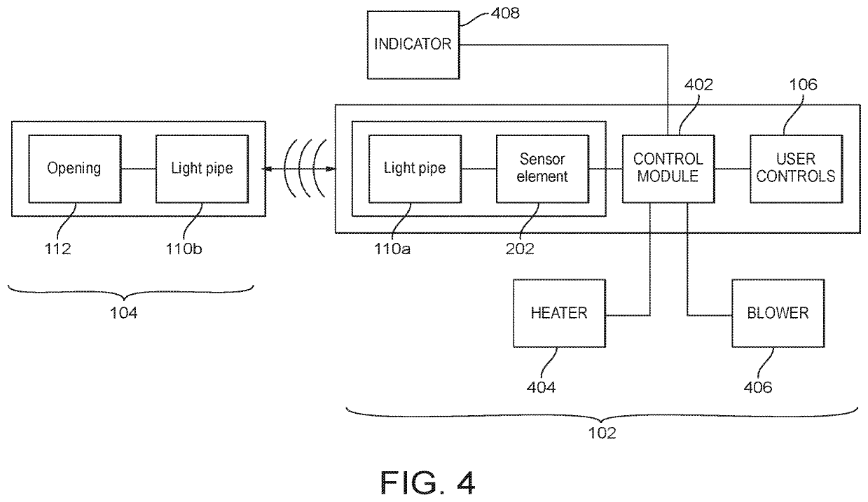

Now referring to FIG. 4, which shows a schematic diagram of haircare appliance according to the present disclosure.

Haircare appliance 100 comprises again a main body 102 attached to an accessory 104. The haircare appliance comprises a control module 402 for controlling the operation of the haircare appliance by e.g., activating and deactivating the heater 404, blower 406 and signalling to a user by indicator 408. User controls or user interface 106 is in communicative connection with control module 402, so that a user may set a desired mode of operation. Attached to the control module 402 is sensor element 202, which in turn is connected to a light pipe 110a. A sensor signal emitted from sensor element 202 propagates through light pipe 110a and into an adjacent light pipe 110b arranged in the accessory 104. The signal is emitted through opening 112 onto an object 208, not depicted in FIG. 4. A sensor reading, e.g., a reflected signal, reflected off object 208 is entering through opening 112 and again propagating through light pipe 110b and 110a towards sensor element 202, which receives the sensor signal by receiver 206, not specifically depicted in FIG. 4 and communicates the sensor reading to the control module 402. Control module 402 may analyse the sensor readings and may adapt the mode of operation of the haircare appliance in reaction to the received sensor signal.

The opening may generally be a through-hole opening without a further element or may alternatively be embodied comprising a further optical element. The optical element may be a substantially transparent cover, e.g., a glass or plastic cover, or may be a focusing element, like a lens to focus the light on a given area.

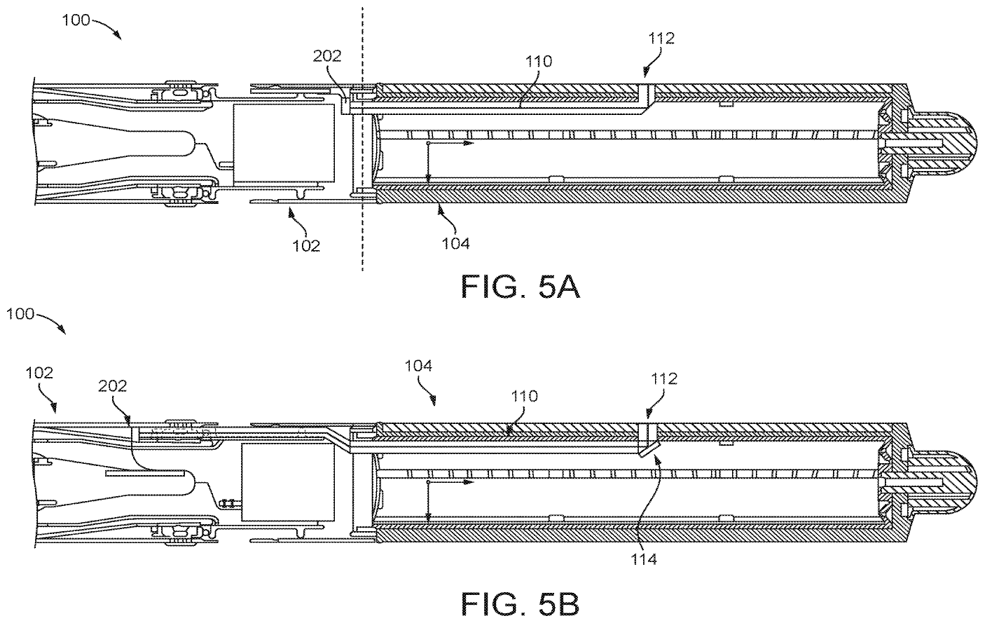

Now referring to FIGS. 5A and 5B, which show cross-sectional views of exemplary embodiments of haircare appliances according to the present disclosure.

FIGS. 5A and 5B show similar haircare appliances 100 as was depicted with regards to FIG. 1. Both haircare appliances 100 comprise a main body 102 and an accessory 104. A sensor element 202 is arranged in the interior of the main body 102. In FIG. 5A, the sensor element 202 is arranged close to the boundary of the main body 102 and the accessory 104, whereas in FIG. 5B, the sensor element 202 is arranged at a distance from the boundary of the main body 102 and the accessory 104, positioned further distal from the boundary. An element for obtaining a sensor reading 110, e.g., a light pipe or light propagating element is provided for connecting the sensor element 202 with the opening 112 in order to obtain a sensor reading from an acquisition region. The acquisition region essentially corresponds with the area or region in close proximity to the opening 112, where a sensor signal originating from the sensor element 202, propagated through the element for obtaining a sensor reading a 110 is exiting from opening 112 onto an object 208. The measurement signal is again acquired through the opening 112 for being propagated back to the sensor element 202 to obtain the sensor reading.

The element for obtaining a sensor reading 110 is embodied differently in the embodiment of FIGS. 5A and 5B. In FIG. 5A, the sensor element 202 is arranged close to the boundary or intersection of the main body 102 and the accessory 104. Here, by attaching the accessory 104, the element for obtaining a sensor reading 110 is brought in close proximity to the sensor element 202, so that signals originating from and measurement signals returning to sensor element 202 are introduced into the element for obtaining a sensor reading simply by placing the sensor element 202 close to the element for obtaining a sensor reading 110. The element for obtaining a sensor reading 110 is substantially completely arranged in accessory 104 in the embodiment of FIG. 5A, with no separate parts being arranged in the main body 102. The element for obtaining a sensor reading 110 is embodied as a light pipe that has at its ends in the area of the opening 112 an integrated 45° cut for redirecting propagated light in a 90° angle to the outside of accessory 104. The element for obtaining a sensor reading 110 in FIG. 5A is thus substantially a one-piece element.

The element for obtaining a sensor reading 110 in FIG. 5B comprises two sections, one section that is arranged between the sensor element 202 and the boundary of main body and accessory in the interior of the main body 102. A further element for obtaining a sensor reading 110, a second part of the light pipe, is arranged in the interior of the accessory between the boundary of main body and accessory and continues to the opening 112. The first part of the light pipe and the second part of the light pipe align so to be in optical communication when the accessory is attached to the main body. In the embodiment of FIG. 5B, the object for obtaining a sensor reading 110 essentially terminates in the interior of the accessory 104 in close proximity to the opening 112, however without being redirected as in the embodiment of FIG. 5A. Rather, a separate optical element 114, e.g., a mirror, is arranged in the vicinity of the end portion of the object for obtaining a sensor reading 110 terminating in the interior of the accessory 104 for redirecting the light propagating through the object for obtaining a sensor reading 110 towards and through the opening 112 into the acquisition region at the exterior of the accessory 104.

The embodiments of FIGS. 5A and 5B obviously can be selectively combined, e.g., it is conceivable to have an integrated 45° cut in the object for obtaining a sensor reading 110 while at the same time the sensor element 202 is spaced distal from the boundary of the main body at the accessory. Preferably, generally speaking, the main body 102 and the accessory 104 may comprise a connection mechanism so that the main body and the accessory can be connected in a reliable and repeatable manner while at the same time assuring proper alignment of elements arranged in the interior of the main body with elements arranged in the accessory. In other words, the connection mechanism may assure that a sensor element 202 in the main body 102 is arranged relative to an element for obtaining a sensor reading 110 in the accessory 104 in a reliable and repeatable manner away connecting an accessory to the main body.

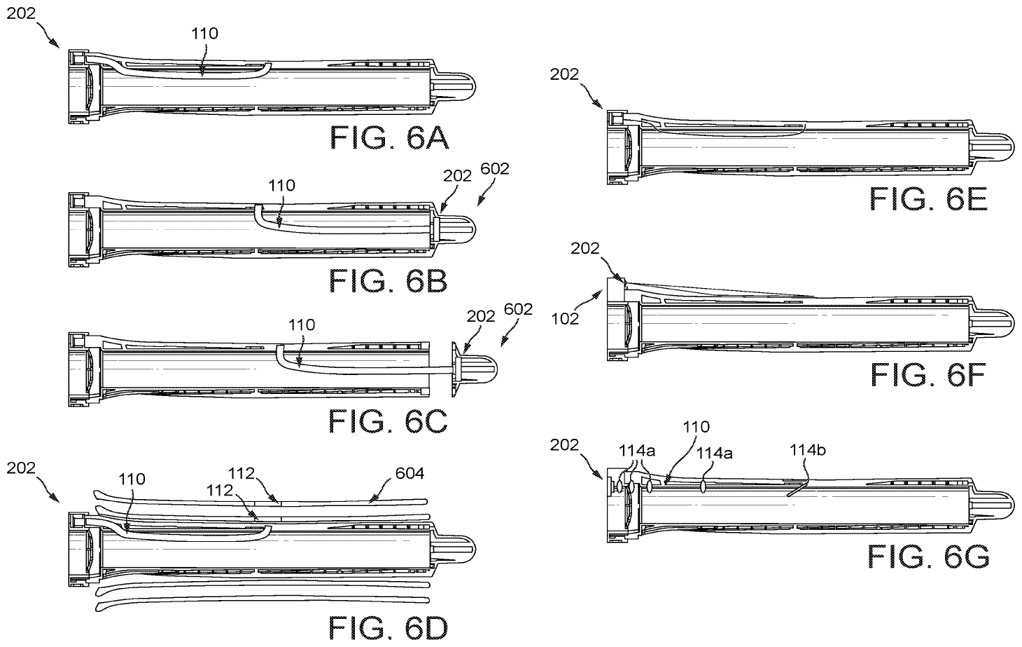

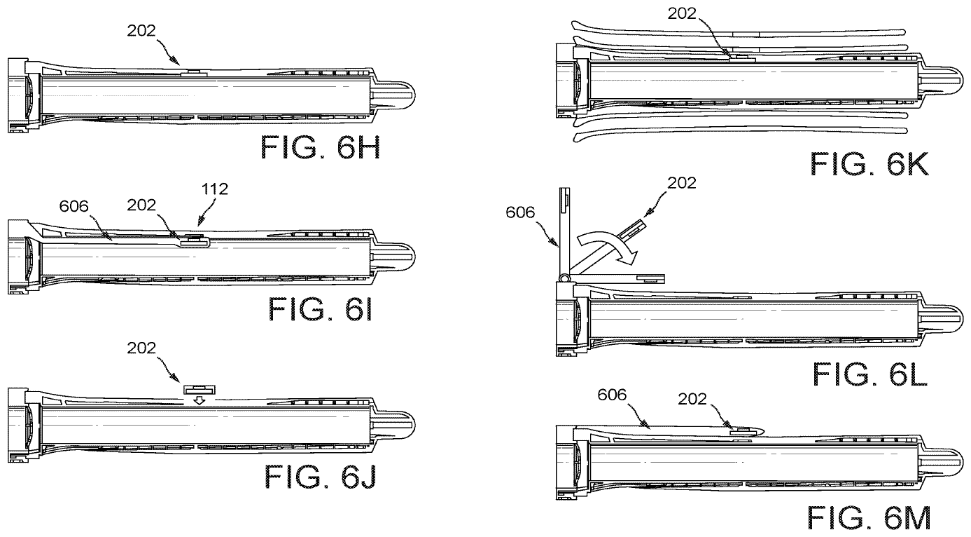

Now referring to FIGS. 6A to 6M, which show cross-sectional views of exemplary embodiments of haircare appliances according to the present disclosure.

FIGS. 6A to 6G show exemplary embodiments where the sensor element is not arranged within the accessory, whereas FIGS. 6H to 6M show embodiments where the sensor element itself is arranged within or in the general region of the accessory.

The embodiment of FIG. 6A is comparable to the embodiments of FIG. 5A, 5B in that an element for obtaining a sensor reading 110, e.g., a light pipe is arranged in the interior of the accessory.

The embodiments of FIGS. 6B and 6C also comprise an element for obtaining a sensor reading 110 in the interior of the accessory, however the sensor element 202 is not arranged within the main body of the haircare appliance but in a tip 602 of the accessory. The tip 602 may be part of the accessory as depicted in FIG. 6B or may itself be removable, i.e., detachable, as is the case in the embodiment of FIG. 6C. The element for obtaining a sensor reading 110 itself may be flexible so that the tip 602 of the accessory can be detached from the accessory and exchanged with a different type of sensor element. In the case of FIG. 6C, a connection mechanism may be provided for connecting the element for obtaining a sensor reading 110 and the sensor element 202, e.g., for removably connecting the sensor element 202 in the tip 602, when connecting a specific tip to the accessory. In other words, by changing the tip 602 and thus the sensor element 202 contained in the tip 602, different types of measurement may be performed. The replacing of the sensor element 202 with tip 602 also allows the exchange of the sensor element in case of a defect without the need to replace the complete accessory. Still further, this allows the exchange of accessories while keeping the sensor element 202 in tip 602, thereby reducing the cost by providing a shared tip 602, as long as tip 602 is attachable to a plurality of different accessories.

In the embodiment of FIG. 6D, the accessory comprises an outer accessory part 604, consisting exemplarily of an inner and an outer shell. Openings 112 to allow the propagation of the sensor signal are provided in the outer accessory part so to allow extending the acquisition region to the outside of the opening 112 on the outer accessory part 604.

In the embodiment of FIG. 6E, the element for obtaining a sensor reading 110 is a glass fibre, thereby reducing the space required in the interior of the accessory.

In the embodiment of FIG. 6F, the sensor element 202 is still arranged at the main body 102, however situated on the exterior of the main body 102 in an elevated manner, thereby overlooking the acquisition region. The sensor element 202 may thus obtain a sensor reading from the acquisition region without the requirement for a specific internal element for obtaining a sensor reading 110 or light pipe.

In the embodiment of FIG. 6G, the element for obtaining a sensor reading 110 is not a dedicated one-piece element but rather consists of a transmission path comprising a plurality of optical elements. Exemplarily, the element for obtaining a sensor reading 110 comprises four lenses 114a for focusing light along the intended travel path between the sensor element 202 and the acquisition region. The sensor signal thus propagates essentially in free air in the interior of the accessory. A further optical element embodied as a mirror element 114b and having a 45° alignment relative to the path of travel of the sensor signal is used to redirect the sensor signal and the measurement signal between the interior of the accessory and the acquisition region.

In the following embodiments 6H to 6M, the connection between the sensor element 102 and the control module 402, for providing energy and communication capability, is not specifically depicted.

In the embodiment of FIG. 6H, the sensor element 202 is integrated into the accessory essentially in the acquisition region. Thereby, it is conceivable that a separate element for obtaining a sensor reading 110 may be simplified, i.e., significantly reduced in size, or may be omitted altogether. In a case where the element for obtaining a sensor reading 110 is simplified, it is conceivable that the element essentially corresponds to a protective cover of the sensor element 202. In other words, it is conceivable that the element for obtaining a sensor reading 110 and the sensor element 202 form an integral, one-piece part.

In the embodiment of FIG. 6I, the sensor element 202 is arranged in the interior of the accessory adjacent to the opening 112, however situated on a holder 606, e.g., an arm extending from the main body 102 into the interior of the accessory 104. In this embodiment, the change of accessories is still easily possible since the sensor element 202 is not attached to the accessory.

In the embodiment of FIG. 6J, the sensor element 202 is integrated into the exterior wall of the accessory but is removable from the accessory. In other words, the sensor element 202 and the accessory are independent from one another so that the sensor element 202 may be removed from the accessory, the accessory may be exchanged with a different type of accessory and the sensor element 202 may be reattached to the newly attached accessory for continued use.

In the embodiment of FIG. 6K, which corresponds in outer structure to the embodiment of FIG. 6D, the sensor element 202 again is integrated into the accessory as depicted in the embodiment of FIG. 6H.

In the embodiment of FIG. 6L, the sensor element 202 again is attached to the outside of the main body, again using a holder 606. The holder 606 is attached to the outside of the main body so that it can be rotated around an axis or rotation point at the exterior of the main body.

Here, the sensor element may be rotated away (to the left in FIG. 6L) from the acquisition region in order to exchange the accessory or to place hair to be dried appropriately around the exterior of the accessory and subsequently rotate the sensor element 202 by using the holder 606 into place (to the right in FIG. 6L) for taking measurements.

The embodiment of FIG. 6M substantially corresponds to the embodiment of FIG. 6I in that the sensor element 202 is attached to the main body by using a holder 606. In the embodiment of FIG. 6M however, the holder is arranged at the exterior of the accessory. Like in the embodiment of FIG. 6I, the accessory in embodiment of FIG. 6M may be changed while maintaining the sensor element 202 attached to the holder 606.

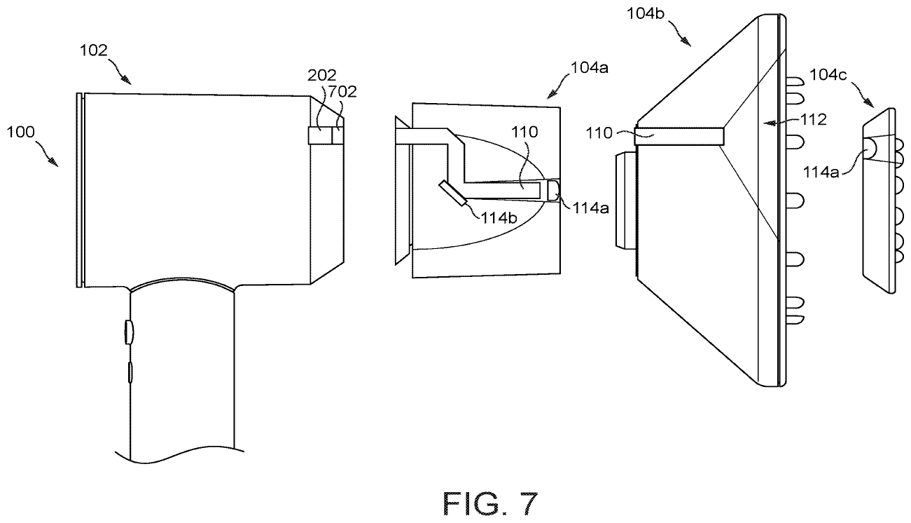

Now referring to FIG. 7, which shows cross-sectional views of an exemplary embodiment of a haircare appliance according to the present disclosure.

The haircare appliance 100 in FIG. 7 is a hair blower, comprising of a main body 102 containing a blower, a heater, and control electronics like a control module. Three different accessories 104a-c are depicted, which can alternatively be attached to the main body 102. The main body 102 comprises a sensor element 202, which is arranged closer to an opening or window 702 at the boundary between the main body and the accessory. The opening or window 702 may be open, may have a transparent cover or may itself be embodied as an element for obtaining a sensor reading 110 or light pipe. Each of the attachments 104a-c comprises an integrated element for obtaining a sensor reading 110 or light pipe, which is aligned with the sensor element 202 when the respective attachment is attached to the main body 102. That way, a sensor signal originating from the sensor element 202 can be transported through the element for obtaining a sensor reading 110 to the acquisition region and a measurement signal in turn can be transported back through the element 110 to the sensor 202.

The specific embodiment of the element for obtaining a sensor reading 110 is dependent on the shape and application of the accessory. For example, the left accessory 104a comprises a light pipe 110 that comprises two right angles so that the acquisition region is arranged centrally with regard to the accessory 104a while the sensor element 202 itself is arranged off centre, e.g., at the outer circumference of the main body. One 90° bend is embodied by using an optical element 114b or mirror, while the other 90° bend is embodied as a 45° cut in the material of the light pipe 110. In other words, the redirecting of the sensor signals corresponds to the two embodiments described with regards to FIGS. 5A and 5B in the area of the opening adjacent to the acquisition region. A further optical element 114a or lens is provided with the accessory 104a, e.g., for focusing the sensor signal onto the acquisition region.

The middle accessory 104b essentially comprises an element for obtaining a sensor reading 110 embodied as a straight element without any bends, connecting the sensor element 202 with an opening 112 in the accessory 104b, to connect the sensor element with the acquisition region. The acquisition region may be a spread-out region, exemplified by the light triangle, so that a dedicated optical element like a lens may not be required with the accessory 104b.

The right accessory 104c only comprises a single optical element 114 a, a lens, arranged adjacent to the sensor element 202 when accessory 104c is attached to the main body. Due to the close proximity of the sensor element 202 and the optical element 114a, no dedicated element for obtaining a sensor reading 110 may be necessary. Alternatively, or additionally, the window 702 may itself be embodied as an element for obtaining a sensor reading 110 or light pipe, thereby bridging a gap between sensor element 202 and lens 114a.

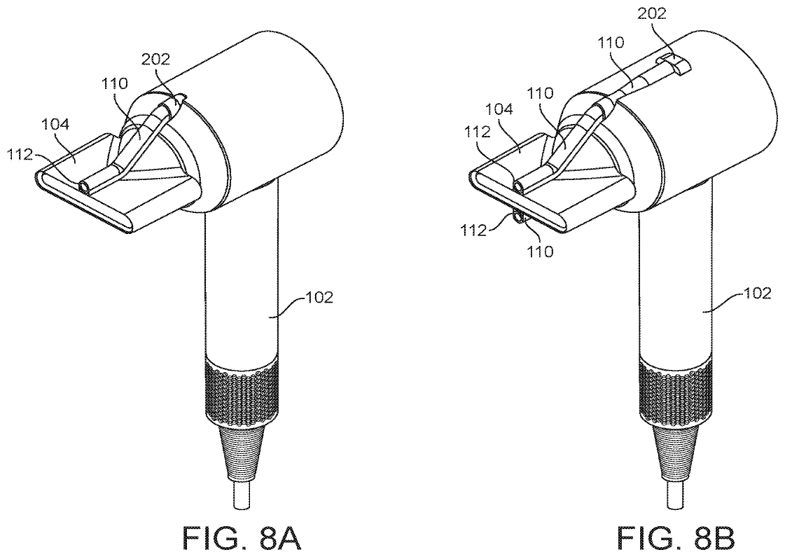

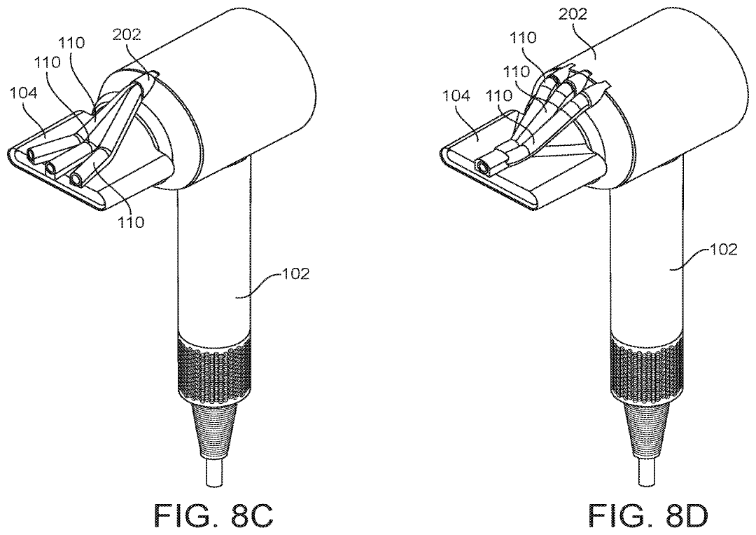

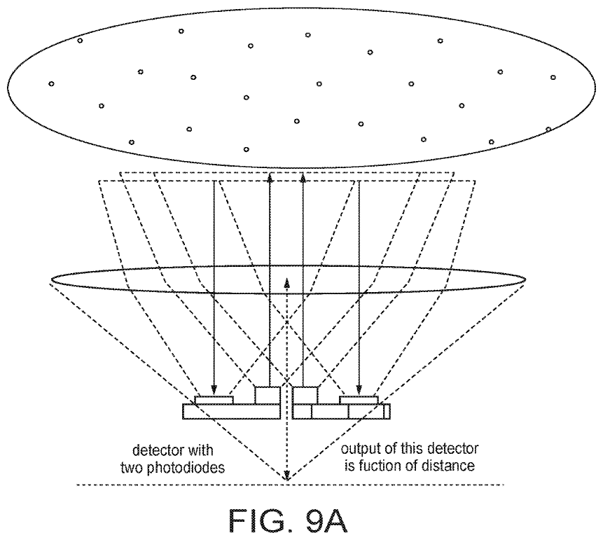

Now referring to FIGS. 8A to 8D, which show views of exemplary embodiments of haircare appliances according to the present disclosure.

The haircare appliance 100 of FIGS. 8A to 8D is essentially comparable to the haircare appliance as depicted with regard to FIG. 7, while the accessory depicted in FIGS. 8A to 8D is essentially comparable to the accessory 104a of FIG. 7.

In FIG. 8A, the haircare appliance 100 of FIG. 7 is depicted with accessory 104a. Sensor element 202 is arranged at the outer peripheral edge of the main body 102, continuing with an element for obtaining a sensor reading 110 along the outer edge of accessory 104, until arriving at the opening 112 adjacent to the acquisition region. The sensor element 202 may thus provide a sensor signal that is propagated through the light pipe 110 onto the acquisition region where a measurement signal is taken and propagated back through the light pipe 110 to the sensor element 202.

In FIG. 8B, the main body 102 comprises a sensor element 202 arranged centrally at the top side of the haircare appliance 100. A first element for obtaining a sensor reading or light pipe 110 is provided at the main body 102, running from the sensor element 202 to accessory 104, where it continues with a further element for obtaining a sensor reading 110/light pipe running along the outer surface of the accessory 104. A first opening 112 of the element for obtaining a sensor reading 110 is depicted at the top side of the accessory 104 to release the sensor signal onto the acquisition region and for obtaining the measurement signal and propagating back to the sensor 202. A second opening 112 is depicted at the bottom side of the accessory connected to a second element for obtaining a sensor reading/light pipe 110 that this running along the bottom side of the accessory. It is conceivable that the bottom light pipe is connected to a separate sensor element not depicted in FIG. 8B or may connect to the same sensor element 202 shown, by an appropriate element for obtaining a sensor reading or light pipe 110 arranged in the interior of the haircare appliance 100. In case of a separate sensor element, it is conceivable that a different measurement value is taken by the bottom light pipe compared to the top light pipe, or in other words that the second sensor element is measuring a different property than the sensor element 202 shown in FIG. 8B. In case both openings are attached to the same sensor element 202 or to two similar sensor elements 202, the acquisition region may be increased.

In FIG. 8C, a single sensor element 202 is connected to exemplary three elements for obtaining a sensor reading 110, which run along the top side of the accessory and angled relative to one another, thereby potentially increasing the total acquisition region. It is conceivable that the sensor element acquires measurement information from the respective individual acquisition regions substantially concurrently or in a defined consecutive succession, i.e., one after the other.

Contrary hereto, in FIG. 8D, a plurality of three sensor elements 202 are connected to a single acquisition region by individual light pipes 110. It is thus conceivable that the individual sensor elements 202 are arranged to measure a different physical property of the same acquisition region. For example, each of the three sensor elements 202 may detect light in a defined wavelength separate to the respective other sensor elements so that substantially simultaneously three different measurement signals can be acquired, each relating to their respective wavelength or frequency band. Alternatively, some or all of the sensor elements 202 may be identical sensor elements and the sensing quality may be increased by using a plurality of identical sensor elements in parallel.

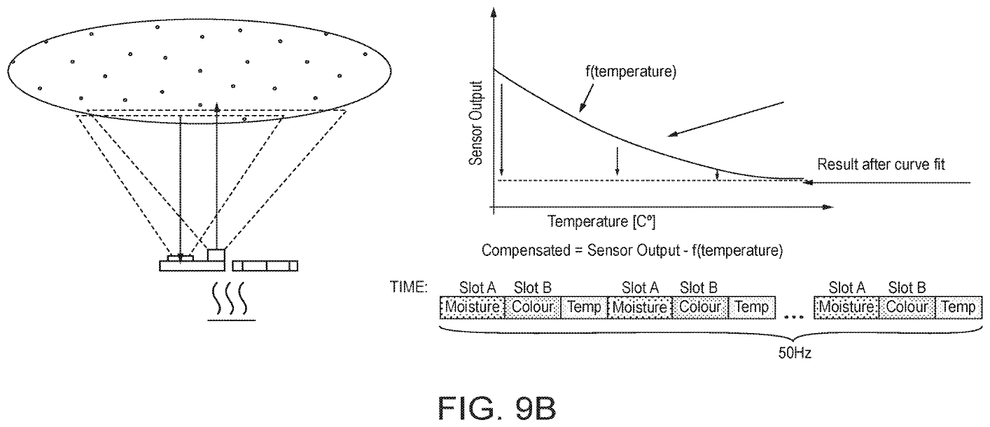

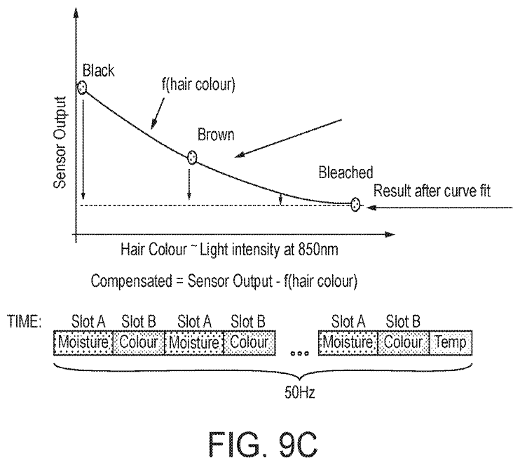

Now referring to FIGS. 9A to 9C, which show exemplary embodiments of measurement compensation according to the present disclosure.

FIG. 9A relates to the detection of a distance to an object in the acquisition region. For example, when considering the haircare appliance of FIG. 8A, it is conceivable that a user may hold the haircare appliance close to the hair to be dried or potentially at a distance. Thus, the hair is a different distance from the opening 112 and is thereby located at a different position within the acquisition region. The distance between the opening 112 and the object may significantly impact any obtainable measurement so that it is beneficial to determine said distance and compensate for a specific current distance. For example, when the hair is close or substantially against the opening 112, ambient light is substantially blocked out when acquiring a sensor measurement. Meanwhile, in a case where the hair is spaced at a distance, e.g., of 5 cm or 10 cm, ambient light may enter the element for obtaining a sensor reading 110/the light pipe 110 and thus it may be necessary for the sensor element 202 to compensate for said additional ambient light. Further, it is conceivable that the measurement may be out of focus in case of an extended distance between the opening 112 and the object 208, so that one outcome of the distance measurement may be to disregard a current measurement, simply because the hair is not within the acquisition region at that measurement time.

The sensor element of the haircare appliance may be embodied to receive distance information that may be used for determining a distance to an object, e.g., hair in the acquisition region. Additionally, or alternatively, a further sensor element may be provided for specifically determining the distance to the object, i.e., hair. Such a distance sensor elements may e.g., be a time-of-flight sensor element determining the hair presence or the distance between the haircare appliance and the hair. The determined distance may only be used to the extent of activating or deactivating the measurement operation or the determined distance may directly influence the determination of the moisture content of the hair by using the determined distance as parameter when calculating the moisture content. Also, the hair presence detection as explained with regards to FIG. 4 may be used as a detection of a distance, in that in case no hair is detected to be present, it may be assumed that the distance is too great to allow a sensible measurement.

FIG. 9B relates to the temperature determination when measuring the sensor signal, and in particular to correction of the acquired sensor signals depending on the determined temperature. In order to determine a temperature of the sensor element or rather a temperature in the vicinity of the sensor element within the housing of the haircare appliance, a further temperature sensor element may be provided in addition to the sensor element for determining the moisture content. The determined temperature may subsequently be employed to compensate measurement values of the sensor element. For example, in a case where the sensor element is operating in a comparably hot environment, the measurement characteristics may be different from the measurement characteristics in a comparably cold environment. This may especially be relevant in a measurement situation with a haircare appliance, since regularly, the temperature span during normal operation of a haircare appliance can be significant. For example, a haircare appliance may operate substantially with unheated air for styling or alternatively with significantly heated air for drying of hair.

Temperature information may not only be derivable from a dedicated temperature sensor but may also be acquired from the general mode of operation or a history of operation. For example, in a case where the haircare appliance is operating for a defined time period without heating, it can be assumed that the temperature in the interior of the haircare appliance does not significantly exceed an ambient temperature. Alternatively, in a case where the haircare appliance has been operating for a defined time period with the heater switched on, it can be assumed that the temperature in the interior of the haircare appliance significantly exceeds ambient temperature. In the latter case, it may be assumed that the temperature in the vicinity of the sensor element may substantially correspond to the temperature of the airflow. Thus, temperature information for controlling the temperature of the haircare appliance may likewise be used as temperature information during the determination of a moisture content.

The right diagram in FIG. 9B exemplifies the relationship between the sensor output at a defined temperature versus the sensor output at a nominal temperature. In particular, the sensor output may increase with an increase in ambient temperature around the sensor element. Thus, without knowledge of the ambient temperature, it cannot be determined with certainty to what extent a certain measurement value is related to the measured property, e.g., light intensity. In case of an increase in sensor output without knowledge about the ambient temperature, the increase may be due to an actual increase in the measured property or alternatively may be due to an increase in the ambient temperature. It is thus preferred to determine a compensated sensor output by removing the influence of the temperature on the sensor output. By using such temperature information, e.g., acquiring current ambient temperature information by a separate temperature sensor, prestored information on the extent of the influence of the respective temperature on the measurement value may be used to compensate the sensor output. E.g., a certain sensor output deviation vs. temperature for given conditions/target measured may be used to generate a lookup table or a curve fit to be applied to the measured sensor output to compensate the measurement value so to zero any offsets due to temperature. Such a curve fit may e.g., be a polynomial type fit or piecewise fit.

Measurements may be taken in a time division multiplex manner, successively acquiring moisture, colour and temperature measurements. Such measurements may e.g., be performed with a frequency of 50 Hz, i.e., one measurement is performed every 20 ms.

FIG. 9C relates to the determination of a melanin content of the hair. In order to determine the melanin content of the hair under treatment, the sensor element may be arranged to base a sensor signal on a spectral band that correlates with a spectral band absorbed by melanin. Thus, the sensor element may detect the melanin content in the hair currently under treatment and subsequently may compensate for the melanin content during the moisture determination. For example, dependent on the melanin content, the sensor element may use different spectral bands for determining the moisture content that are preferably adapted to the specific melanin content so to increase the signal-to-noise ratio of the measurements.

As described with regards to a measurement value deviation due to an ambient temperature, also a different hair colour may influence intensity measurements to the extent that the measurement value may not reliably predict the actual intensity measured. As may be taken from FIG. 9C, the hair colour may influence the sensor output differently. Thus, a hair colour determination may be performed e.g., by light intensity measurement using a wavelength of e.g., 850 nm. The influence of the hair colour on measurements at this wavelength may be predetermined, so that the actual hair colour may be derivable from a measurement performed while operating the haircare appliance. Once the hair colour is determined, the sensor output vs. hair colour for given conditions/temp measured may be compensated by a lookup table or predetermined curve fit that is applied to compensate the measurement value to zero any offsets due to colour. Again, such a curve fit may e.g., be a polynomial type fit or piecewise fit.

Preferably, the sensor output is compensated with regards to a plurality of parameters, e.g., with regards to temperature and hair colour simultaneously. The combination of colour and temperature may lead to multiple curve fits or a 2-dimensional map of temperature and hair colour, where the output is a single value to correct the output of the sensor. This may of course be extended for additional relevant parameters, which are preferably determined when operating the haircare appliance.

Measurements may be taken again in a time division multiplex manner, successively acquiring moisture, colour and temperature measurements. Such measurements may e.g., be performed with a frequency of 50 Hz, i.e., one measurement is performed every 20 ms.

It is to be understood that the invention is not limited to the embodiments described above, and various modifications and improvements may be made without deviating from the concepts described here. Any of the features described above and below may be used separately or in combination with any other features described herein, provided they are not mutually exclusive, and the disclosure extends to and includes all combinations and sub-combinations of one or more features described herein.

Finally, it should be noted that the term “comprising” does not exclude other elements or steps, and that “a” or “one” does not exclude the plural. Elements that are described in relation to different types of embodiments can be combined. Reference signs in the claims shall not be construed as limiting the scope of a claim.

1. A haircare appliance, comprising
a main body comprising a blower for generating an airflow,
wherein the main body comprises an attachment region adapted for connecting an accessory, and
an accessory, comprising an element for obtaining at least one sensor reading at an acquisition region at or around the accessory,
wherein information related to the sensor reading is obtained at the acquisition region;
wherein the accessory is attachable to and/or detachable from the main body at the attachment region,
wherein the accessory, when attached to the main body, is adapted to receive the airflow from the blower, and
wherein the accessory is adapted to discharge the received airflow towards the hair of a user,
wherein the information related to the sensor reading is transmitted from the acquisition region to a sensor element, and
wherein the information is transmitted wirelessly.

2. The haircare appliance according to claim 1, wherein the element for obtaining at least one sensor reading is an element for wirelessly transmitting the information related to the sensor reading from the acquisition region to the sensor element.

3. The haircare appliance according to claim 1,
wherein the element for obtaining at least one sensor reading is the sensor element.

4. The haircare appliance according claim 1,
wherein the sensor element is arranged in the main body, or
wherein the sensor element is arranged in the accessory, or
wherein the sensor element is arranged in an intermediary element arranged between the main body and the accessory.

5. The haircare appliance according claim 1,
wherein the sensor element is functionally associated with the element for obtaining at least one sensor reading, so that the information related to the sensor reading transmitted from the acquisition region is receivable by the sensor element.

6. The haircare appliance according to claim 1,
wherein the sensor element is an active sensor element for actively generating a sensor reading initiation signal that is transmitted to the accessory to initiate the information related to the sensor reading.

7. The haircare appliance according to claim 1,
wherein the sensor reading initiation signal is transmitted to the acquisition region through the element for obtaining at least one sensor reading, and/or
wherein the sensor reading initiation signal is transmitted to the acquisition region through a further transmission element.

8. The haircare appliance according to claim 1,
wherein the haircare appliance comprises a plurality of acquisition regions and/or a plurality of sensor elements,
wherein the haircare appliance comprises a plurality of elements for obtaining at least one sensor reading and/or a plurality of further transmission elements, and
wherein the plurality of elements for obtaining at least one sensor reading and/or the plurality of further transmission elements connect the plurality of acquisition regions and/or the plurality of sensor elements.

9. The haircare appliance according to claim 1,
wherein sensor element is adapted to generate the sensor reading initiation signal,
wherein the sensor reading initiation signal is transmitted to the acquisition region,
wherein the sensor reading initiation signal interacts with an object at or around the acquisition region for generating the information related to the sensor reading,
wherein the element for obtaining at least one sensor reading is adapted to transmit the generated information related to the sensor reading to the sensor element,
wherein the sensor element is adapted to receive the information related to the sensor reading, and
wherein the sensor element is adapted to process the received information related to the sensor reading to obtain the sensor reading.

10. The haircare appliance according to claim 1,
wherein the element for obtaining at least one sensor reading is a light propagating element; and
wherein the light propagating element is arranged for transmitting the information related to the sensor reading as electromagnetic radiation from the acquisition region to the sensor element.

11. The haircare appliance according to claim 1,
wherein the element for obtaining at least one sensor reading is at least one element or arrangement out of the group consisting of an optical element for collecting, transporting/guiding and distributing light, a lens, a mirror, a hollow prismatic pipe, a light rod, a mirrored light pipe, an optical fibre and an optical filter.

12. The haircare appliance according to claim 1,
wherein the sensor reading is a sensor reading of an attribute of hair treatment currently treated by the haircare appliance, and
wherein the attribute is indicative of at least one parameter out of the group consisting of a moisture level of the hair, a hair moisture, a temperature of the hair, a distance between the accessory and the hair, a hair presence, an ambient temperature, an ambient light level, a hair colour and a hair melanin content.

13. The haircare appliance according to claim 1,
wherein the sensor element is arranged in the haircare appliance so to be not exposed to the physical operation of the blower, the airflow of the blower and/or heat provided by a heating element for heating the airflow.

14. The haircare appliance according to claim 1,
wherein the operation of the haircare appliance is adaptable dependent on the sensor reading.

15. The haircare appliance according to claim 1,
wherein the powering of the blower, the powering of a heater associated with the blower and/or the powering of the accessory is dependent on the sensor reading.
